(19) United States
(12) Patent Application Publication
(10) Pub. No.: US 2011/0282122 A1
(43) Pub. Date: Nov. 17, 2011

(54) MOLECULAR SIEVE, CATALYST, AND/OR A PROCESS RELATING THERETO

(75) Inventors: Hui Wang, Des Plaines, IL (US); Paula L. Bogdan, Mount Prospect, IL (US); Richard R. Willis, Cary, IL (US)

(73) Assignee: UOP, LLC, Des Plaines, IL (US)

(21) Appl. No.: 12/780,521

(22) Filed: May 14, 2010

(51) Int. Cl. C07C 4/12; C01B 39/02; B01J 29/06; C01B 39/00

(52) U.S. Cl. 585/486; 423/700; 502/60; 502/64

(57) ABSTRACT

One exemplary embodiment can be a molecular sieve. The molecular sieve can include one or more crystals. The molecular sieve can have an external surface area of no more than about 20 m²/g.

### FIELD OF THE INVENTION

This invention generally relates to a molecular sieve, a catalyst, and/or a process utilizing at least one of the molecular sieve and/or catalyst.

### DESCRIPTION OF THE RELATED ART

Generally, a zeolite is a molecular sieve that can be utilized for catalyzing reactions or adsorbing substances. Often, the zeolite can be used in conjunction with other materials to facilitate reactions. As such, the zeolite can either be used as the catalytic material, or be used as a support for, e.g., one or more metals that are deposited thereon.

Often, zeolites can have well-defined micropores that allow molecules of a given size to pass there-through. As a result, this intrinsic property can enable zeolite materials to achieve the desired reaction selectivity for converting feeds into desired products.

Generally, it is desirable to have a catalyst with a maximized surface area to facilitate chemical reactions. However, if the catalytic material is too reactive, it can create unwanted side-reactions. As an example, often in the conversion of a C8 feed to xylenes competing reactions can occur, such as the production of ethylbenzene. Consequently, it is desirable to have a catalyst that is effective without facilitating unwanted side-reactions. In addition, the production of zeolites often utilize templating agents. It would be desirable to utilize templating agents that are inexpensive and disposable in an environmentally friendly manner. Hence, there would be a desire to provide a zeolite that may minimize unwanted side-reactions, and inexpensively manufactured in an environmentally suitable manner.

### SUMMARY OF THE INVENTION

One exemplary embodiment can be a molecular sieve. The molecular sieve can include one or more crystals. The molecular sieve can have an external surface area of no more than about 20 m²/g.

Another exemplary embodiment may be a catalyst. The catalyst can include a molecular sieve including one or more crystals and having an external surface area of no more than about 20 m²/g, and a binder.

Yet a further exemplary embodiment can include a process for dealkylating ethylbenzene. The process can include passing a stream having ethylbenzene over an effective amount of a catalyst. Generally, the catalyst includes a molecular sieve having one or more crystals and an external surface area of no more than about 20 m²/g, and a binder.

Therefore, the embodiments disclosed herein can provide a relatively small surface area, which can minimize unwanted side-reactions, such as having an external surface area of no more than about 20 m²/g. Although not wanting to be bound by scientific theory, it is believed that such crystal structures can minimize unwanted side-reactions by directing the catalyst activity to its micropores.

Moreover, there is a desire to produce such zeolites with an inexpensive templating agent that can be disposed of in an environmentally friendly manner. As such, the embodiments disclosed herein can provide templating agents that are easily recovered and reused.

### DEFINITIONS

As used herein, “loss on ignition” may be abbreviated “LOI” and be determined by ASTM D7348-08.

As used herein, the terms “absorbent” and “absorber” include, respectively, an adsorbent and an adsorber, and relates, but is not limited to, absorption, and/or adsorption.

As used herein, the terms “tri-methyl benzene” may be abbreviated “TMB”, “ethylbenzene” may be abbreviated “EB”, “para-xylene” may be abbreviated “pX”, “total xylenes” may be abbreviated “X”, “gram” may be abbreviated “g”, and “meter-squared” may be abbreviated “m²”.

As used herein, the units of BET surface area, external surface area, and micropore area can be in meters-squared per gram, and abbreviated “m²/g”. External surface area and BET surface area can be calculated by ASTM D1993-03 (2008). With respect to the external surface area, the external surface area is equal to the t-area and is calculated by the following formula at paragraph 11.14 of ASTM D1993-03 (2008):

t-area=(Slope of t-plot)*(15.47/0.975)

With respect to the BET surface area, the BET surface area can be calculated at paragraph 11.9 of ASTM D1993-03 (2008) as follows:

BET surface area=(4.353)*(Volume of Adsorbate)

As used herein, the unit of micropore volume can be cubic centimeter per gram, and abbreviated “cc/g.”

### BRIEF DESCRIPTION OF THE DRAWINGS

FIG. 1 is an SEM photograph of crystals of one exemplary zeolite.

FIG. 2 is an SEM photograph of crystals of another exemplary zeolite.

FIG. 3 is an SEM photograph of crystals of a further exemplary zeolite.

FIG. 4 is an SEM photograph of crystals of an additional exemplary zeolite.

FIG. 5 is an SEM photograph of crystals of yet another exemplary zeolite.

FIG. 6 is an SEM photograph of crystals of still another exemplary zeolite.

FIG. 7 is a graphical depiction comparing the CPS intensity versus two-theta degree.

FIG. 8 is a graphical depiction comparing net toluene and TMB yield versus EB conversion for one set of samples.

FIG. 9 is a graphical depiction comparing net toluene and TMB yield versus EB conversion for another set of samples.

FIG. 10 is a graphical depiction comparing the ratio of pX/X yield versus EB conversion.

FIG. 11 is a graphical depicture of xylene loss versus EB conversion for another set of samples.

FIG. 12 is a graphical depiction of net toluene and TMB yield versus EB conversion.

### DETAILED DESCRIPTION

In preparing an MFI zeolite (also referred to as a ZSM-5 zeolite), the utilized templating agent can have a relatively high vapor pressure, and as such, create flammability hazards. As such, not only is there a desire to provide a zeolite with a more targeted catalyzing reaction, but also a manufacturing method that minimizes dangers associated with high vapor pressure by selecting templating agents with a relatively low vapor pressure.

Generally, the embodiments disclosed herein provide a method of combining an alumina, water, and silica to form a homogenized mixture for making a zeolite. Afterwards, a templating agent can be added to the mixture. Thus, most of the ingredients can be mixed in a liquid form alone before adding in the templating agent. Typically, no seeds of zeolites are utilized.

As a result, the product can have a relatively small external surface area, such as no more than about 20 m²/g, preferably no more than about 12 m²/g, and optimally no more than about 8 m²/g. Preferably, the molecular sieve, typically an MFI zeolite, can be combined with a binder. The binder can include at least one of titania, zirconia, alumina, magnesia, thoria, beryllia, quartz, aluminum phosphate, and silica. Preferably, the binder includes alumina, aluminum phosphate, or silica. Thus, the molecular sieve and binder can comprise a support, which can be impregnated with one or other materials, such as a metal, to form a catalyst, or comprise a catalyst itself. The support or catalyst can include about 1-about 99%, by weight, and preferably about 25-about 75%, by weight, of the molecular sieve based on the weight of the catalyst. Thus, the support or catalyst can include, consist essentially of, or consist of the molecular sieve and the binder.

An optional metal component can be a noble metal component, and may include an optional base metal modifier component in addition to the noble metal or in place of the noble metal. The noble metal may be a platinum-group metal of platinum, palladium, rhodium, ruthenium, osmium, or iridium. The base metal can be of rhenium, tin, germanium, lead, cobalt, nickel, indium, gallium, zinc, uranium, dysprosium, thallium, or a mixture thereof. The base metal may be combined with another base metal, or with a noble metal. Suitable total metal amounts in the isomerization catalyst range from about 0.01-about 10%, preferably from about 0.01-about 3%, by weight. If a noble metal is included, typically the noble metal, e.g., platinum or palladium, can be in an amount of about 0.01-about 2.0%, by weight, preferably about 0.01-about 0.1%, by weight, and optimally about 0.02-about 0.06%, by weight. Suitable zeolite amounts in the catalyst can range from about 1-about 99%, preferably about 10-about 90%, and more preferably about 25-about 75%, by weight. The balance of the catalyst is composed of inorganic oxide binder.

Generally, the materials can be combined at any suitable conditions, such as at room temperature and pressure. Typically, the temperature can be about 10-about 50° C. and the pressure can be about 90-about 130 kPa. Generally, an alumina is dissolved in water and stirred, and then silica can be added and homogenized. Once homogenized, a templating agent can be added and the mixture stirred and heated for about 2-about 5 days, preferably about 3-about 5 days. The heating can be at a temperature of about 120-about 300° C., preferably about 150-about 200° C. Heating can be done at any suitable pressure, such as about 90-about 1,000 kPa, preferably about 90-about 110 kPa.

Typically, the alumina can be any commercially available alumina. In one exemplary embodiment, the alumina may include about 15%, by weight, sodium, about 12%, by weight, aluminum, and have a loss of ignition of about 57%. In addition, any suitable silica can be added. In one exemplary embodiment, the silica may include about 0.5%, by weight, sodium, about 40%, by weight, silica, about 0.04%, by weight, aluminum, and have a loss on ignition of about 9.53%. The templating agent can be at least one of ethylamine, n-propylamine, n-butylamine, ethylene diamine, triethylamine, and hexamine diamine. Preferably, the templating agent is n-propylamine or n-butylamine. These templating agents can be used alone or in combination, and are typically selected due to their relatively low vapor pressure.

Afterwards, the zeolite may be formed and combined with a binder to be a support of a catalyst. Usually, an extrusion aid may be utilized, such as at least one of sesbania powder, a starch, an activated carbon, a polyethylene glycol, a polyacrylamide, or a polyalcohol in an amount of about 0.1-about 4%, by weight, based on the weight of the precursor mixture. Typically, the binder and zeolite can be formed into any suitable catalyst shape, such as disclosed in, e.g., U.S. Pat. No. 7,601,330 B2.

Generally, a catalyst, including the molecular sieve as prepared by the embodiments described herein, can be used for any suitable process, such as converting an alkyl aromatic feed or dealkylating ethylbenzene. Particularly, often a mixed alkyl aromatic feed, including meta-, ortho-, and para-xylene along with toluene and ethylbenzene can be isomerized to form one or more desired alkyl aromatics, typically para-xylene. As a result, it is generally desirable to dealkylate ethylbenzene to provide more feedstock, such as benzene, that can be alkylated into other desired products. Usually, the process can include passing a stream including ethylbenzene in the presence of an effective amount of catalyst where the reactions take place. Generally, the reactions can take place at any suitable conditions including a temperature of about 200-about 600° C. and a pressure generally from about 100 kPa-about 5 MPa. The weight hourly space velocity with respect to the feed mixture can be about 0.5-about 50 hr⁻¹ and a hydrogen-to-hydrocarbon mole ratio of about 0.5:1-about 10:1.

Thus, the embodiments as disclosed herein can convert the alkyl aromatic feed without producing undesired side-reactions, such as the conversion of xylenes to trimethylbenzene and toluene.

### ILLUSTRATIVE EMBODIMENTS

The following examples are intended to further illustrate the subject sieves and catalysts. These illustrations of embodiments of the invention are not meant to limit the claims of this invention to the particular details of these examples. These examples are based on engineering calculations and actual operating experience with similar processes. All the examples utilize a mixer sold under the trade designation MULLER by Kercher Industries Inc. of Lebanon, Pa., unless otherwise indicated. The amounts of compounds, such as xylene and ethylbenzene, can be measured with an HP5890 gas chromatograph sold by Hewlett-Packard Company of Palo Alto, Calif. or a gas chromatograph sold under the trade designation Agilent 7890A, by Agilent Technologies of Wilmington, Del.

### Example 1

An exemplary MFI zeolite is initially made by dissolving 20 grams of liquid sodium aluminate into 750 grams of distilled water in a 2-liter beaker. Typically, the liquid sodium aluminate includes 14.8%, by weight, sodium, 12.2%, by weight, aluminum, and have an LOI of 57.01%, by weight. After vigorous overhead stirring, 260 grams of silica are added. The silica includes 0.44%, by weight, sodium, 46.3%, by weight, silicon, and 0.035%, by weight, aluminum. The LOI of the silica is 9.53%, by weight. After homogenization, 285 grams of a templating agent, namely n-butylamine, are added. Following at least 30 minutes of stirring, the reaction gel is transferred into a 2-liter stainless-steel autoclave and heated at about 175° C. for five days.

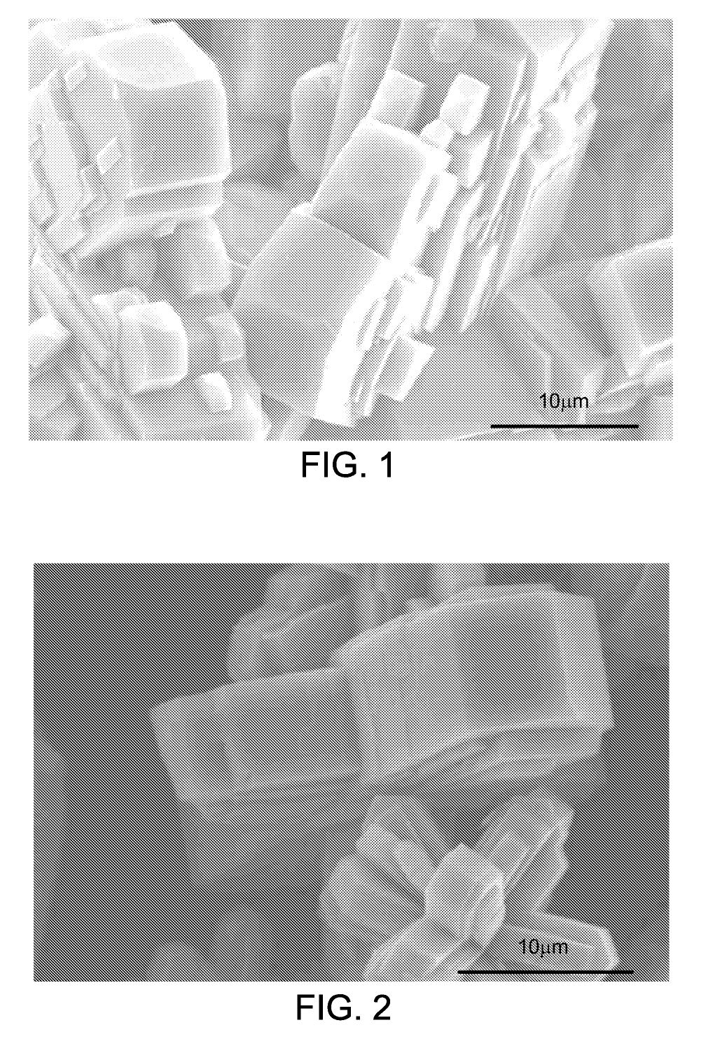

Subsequent to filtering and ion exchanging with ammonium nitrate, the resulting MFI zeolite can include 1.05%, by weight, aluminum and 40.7%, by weight, silicon, an Si/Al₂ ratio of about 75, a BET surface area of 335 m²/g, a micropore area of 323 m²/g, a micropore volume of 0.166 cc/g, and an LOI of 5.95%. Crystals of the MFI zeolite are depicted in FIG. 1.

### Example 2

Another MFI zeolite is initially made by dissolving 15.9 grams of liquid sodium aluminate from Example 1 into 750 grams of distilled water in a 2-liter plastic beaker. Under vigorous stirring, 250 grams of silica from Example 1 are added. After homogenization, 285 grams of n-butylamine are added. Following at least 30 minutes of stirring, the reaction gel is transferred into a 2-liter stainless-steel autoclave and heated at 175° C. for five days. Subsequent to filtering and ion exchanging with ammonium nitrate, the MFI zeolite has 0.89%, by weight, aluminum and 46%, by weight, silicon and an Si/Al₂ ratio of about 100. Crystals of the MFI zeolite are depicted in FIG. 2.

### Example 3

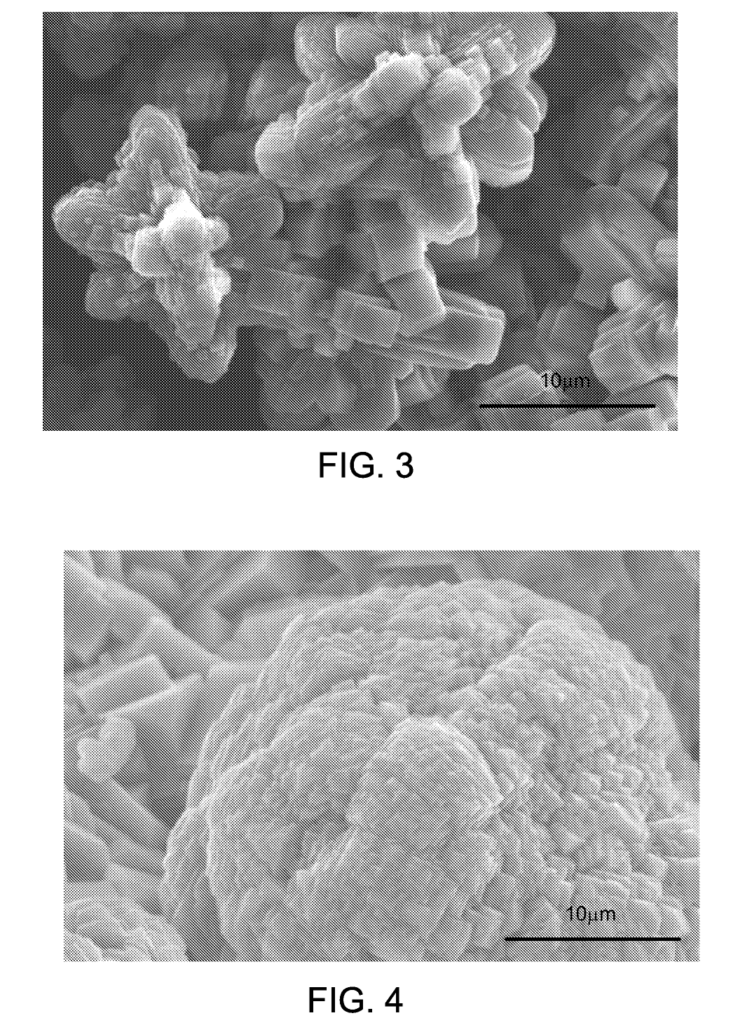

A further MFI zeolite is initially made by dissolving 26.9 grams of liquid sodium aluminate from Example 1 into 750 grams of distilled water in a 2-liter plastic beaker. Under vigorous stirring, 250 grams of silica from Example 1 are added. After homogenization, 285 grams of n-butylamine are added to the above mixture. Following at least 30 minutes of stirring, the reaction gel is transferred into a 2-liter stainless-steel autoclave and heated at 175° C. for five days. Subsequent to filtering and ion exchanging with ammonium nitrate, the MFI zeolite has 1.52%, by weight, aluminum and 45.5%, by weight, silicon, and an Si/Al₂ of about 58. Crystals of the MFI zeolite are depicted in FIG. 3.

### Example 4

Yet another MFI zeolite is initially made by dissolving 3.9 grams of liquid sodium aluminate from Example 1 into 90 grams of distilled water in a plastic bottle. Under vigorous stirring, 30 grams of silica from Example 1 are added. After homogenization, 36 grams of n-butylamine are added to the mixture. Following at least 30 minutes of stirring, the reaction gel is transferred into Parr bombs and heated at 175° C. for five days. The resulting MFI zeolite can have an Si/Al₂ of about 50. Crystals of the MFI zeolite are depicted in FIG. 4.

### Example 5

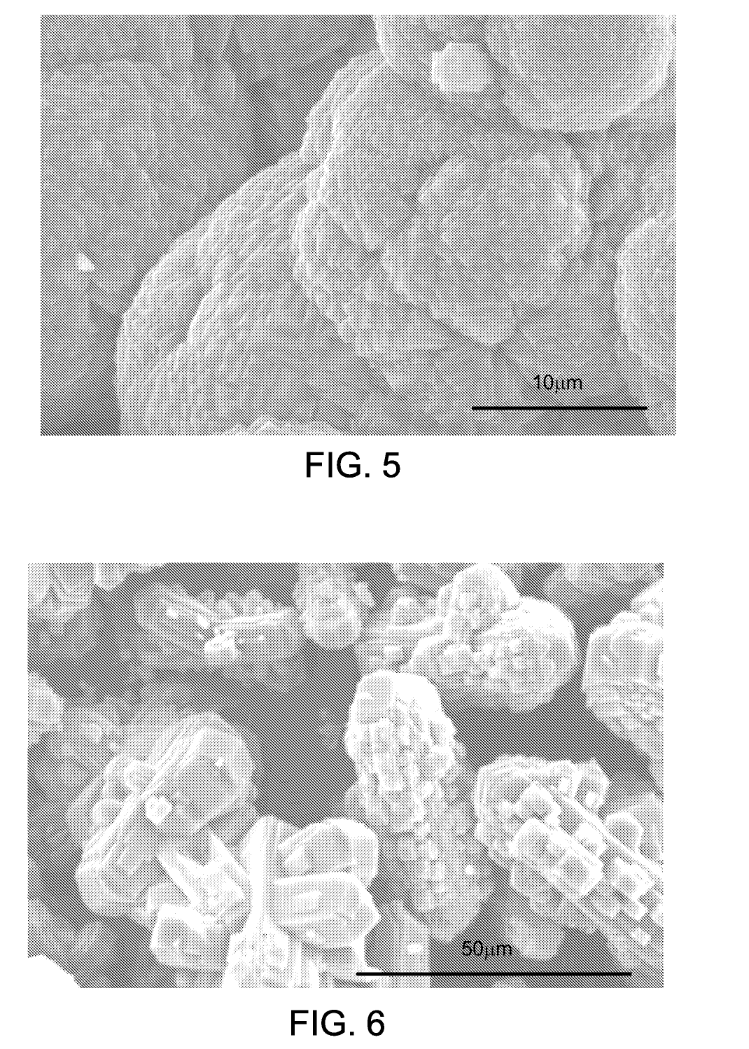

A still further MFI zeolite is initially made by dissolving 4.9 grams of liquid sodium aluminate from Example 1 into 90 grams of distilled water in a plastic bottle. Under vigorous stirring, 30 grams of silica from Example 1 are added. After homogenization, 36 grams of n-butylamine are added to the above mixture. After at least 30 minutes of stirring, the reaction gel is transferred into Parr bombs and heated at 175° C. for five days. Generally, the MFI zeolite can have an Si/Al₂ ratio of about 40. Crystals of the MFI zeolite are depicted in FIG. 5.

### Example 6

Still another MFI zeolite is initially made by dissolving 2.4 grams of liquid sodium aluminate from Example 1 into distilled water in an amount of 90 grams in a plastic bottle. Under vigorous stirring, 30 grams of silica from Example 1 are added. After homogenization, 36 grams of n-propylene are added to the mixture. Following at least 30 minutes of stirring, the reaction gel is transferred into Parr bombs and heated at 175° C. for five days. The resulting MFI zeolite can have an Si/Al₂ ratio of about 75. Crystals of the MFI zeolite are depicted in FIG. 6.

In the Table 1 below, the silica to alumina ratio, BET total surface area, and external surface area of the samples from Examples 1-6 are summarized:

TABLE 1

| | | BET total surface area | External surface area |
|---|---|---|---|
| Example | Si/Al₂ ratio | m²/g | m²/g |
| 1 | 75 | 335 | 12 |
| 2 | 100 | 341 | 9 |
| 3 | 58 | 347 | 8 |
| 4 | 50 | 342 | 10 |
| 5 | 40 | 345 | 13 |
| 6 | 75 | 334 | 7 |

Several exemplary supports or catalysts are prepared from the exemplary molecular sieves.

### Example 7

13.8 grams of the MFI zeolite from Example 1 are mixed using a mortar and pestle with 17.5 grams of a commercially available colloidal silica having 40%, by weight, silica suspended in water sold under the trade designation LUDOX-40 by E. I. Du Pont De Nemours and Company of Wilmington, Del. After air drying, the solid product is calcined at 600° C. for four hours and then crushed through a screen having 8 wires per linear centimeter by 16 wires per linear centimeter. The final product contains 65%, by weight, MFI zeolite and 35%, by weight, amorphous silica.

### Example 8

100 grams of the MFI zeolite from Example 1 are mixed with 56.15 grams of a commercially available silica having LOI of 9.53% sold under the trade designation ULTRASIL by Evonik Degussa GmbH of Evonik Industries AG of Essen, Germany and 105 grams of distilled water. After mixing for 30 minutes, the dough is extruded into 0.16 centimeter trilobe. Following air-drying and calcination at 600° C. for four hours, a final product containing 65%, by weight, MFI zeolite (with Si/Al₂ ratio of about 75) and 35%, by weight, amorphous silica is obtained.

### Example 9

100 grams of the MFI zeolite from Example 2 are mixed with 56.15 grams of a commercially available silica having LOI of 9.53% as described in Example 8, and 98 grams of distilled water in a mixer. After mixing for 30 minutes, the dough is extruded into a 0.16 centimeter trilobe. Following air-drying and calcination at 600° C. for four hours, a final product containing 65%, by weight, MFI zeolite (with Si/Al₂ ratio of about 100) and 35%, by weight, amorphous silica is obtained.

At least some of the supports and/or catalysts prepared above are tested. These tests include toluene transalkylation. This test is conducted at a variable temperature, in which a parameter is the pX/X ratio. This is the ratio of para-xylene, by weight, over the total xylenes, by weight, in a product at 500° C. by the formula:

pX/X=(para-xylene/(para-xylene+meta-xylene+ortho-xylene))

In this test, a feed consisting of a nitrogen carrier gas saturated with a vapor of toluene at 0° C. can be provided to a microreactor. The procedure can include pretreating the molecular sieve or catalyst at a temperature of 550° C. and a pressure of 101 kPa, i.e., atmospheric pressure, for 60 minutes in nitrogen. The catalyst can pass through a screen having 8 wires per linear centimeter by 16 wires per linear centimeter. Afterwards, the testing temperature can vary from 400-500° C. over a time period of 500 minutes with a ramping of temperature from 0-400° C. during the first 100 minutes. Generally, the temperature can average about 450° C. from 100-500 minutes with a catalyst load of 250 milligrams. The feed can flow at a constant 50 cc/min. Products are measured with an HP5890 gas chromatograph sold by Hewlett-Packard Company of Palo Alto, Calif.

In another type of evaluation for catalyst absent of deposited metals, about 1 gram of catalyst is loaded into a fixed bed reactor. A feed and hydrogen are introduced to the reactor to contact the catalyst. The feed is 60% meta-xylene, 25% ortho-xylene, and 15% ethylbenzene, by weight. The feed is pumped at 10 weight hourly space velocity based on the amount of zeolite, and the hydrogen to hydrocarbon ratio is 4. The reactor operates at about 786 kPa absolute and each catalyst is tested at temperatures 375° C., 385° C., and 395° C. The 1 gram of catalyst is introduced with 0.4 gram of particles of a size sufficient to pass through a screen having 6 wires per linear centimeter by 8 wires per linear centimeter to form a physical mixture. The particles include 0.3%, by weight, platinum on alumina catalyst modified with 0.6%, by weight, indium and 0.3%, by weight, tin in accordance with Example III in U.S. Pat. No. 6,048,449 sufficient to achieve 17.5 weight hourly space velocity on this component.

The net toluene (may be abbreviated “TOL”) and TMB yield can be the difference in percentage, by weight, of toluene and TMB in the product compared to the feed. Net toluene and TMB yield are measured with an HP5890 gas chromatograph sold by Hewlett-Packard Company of Palo Alto, Calif.

The xylene loss (may be abbreviated “XL”) in percent, by weight, can be determined by the following formula:

Xylene loss=(Xylene(feed)-Xylene(product))/(Xylene(feed))×100

The ethylbenzene conversion, by weight, can be determined by dividing the difference, by weight, of the EB in the feed and product by the weight of the EB in the feed:

EB Conversion=((EB in Feed)−(EB in Product))/(EB in Feed)×100

### Example 10

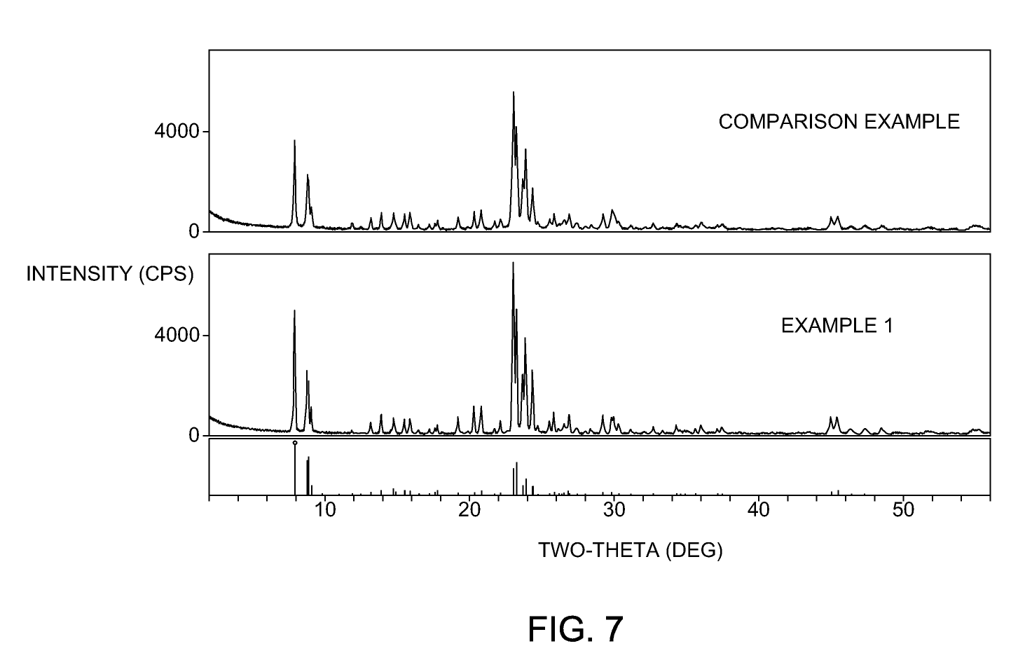

Referring to FIG. 7, an X-ray diffraction pattern of an MFI zeolite with a Si/Al₂ ratio of 38 and an external surface area of 21 m²/g is compared with the molecular sieve prepared according to Example 1. The X-ray diffraction patterns are obtained using an XDS 2000 manufactured by Scintag Inc. of Cupertino, Calif. scanning from 2-56 two theta degree with a scanning speed of 2 degree per minute. As depicted, the X-ray diffraction pattern shows substantially a similar structure with sharper peaks for the zeolite from Example 1. As a consequence, the sharper peaks indicate a larger crystal size.

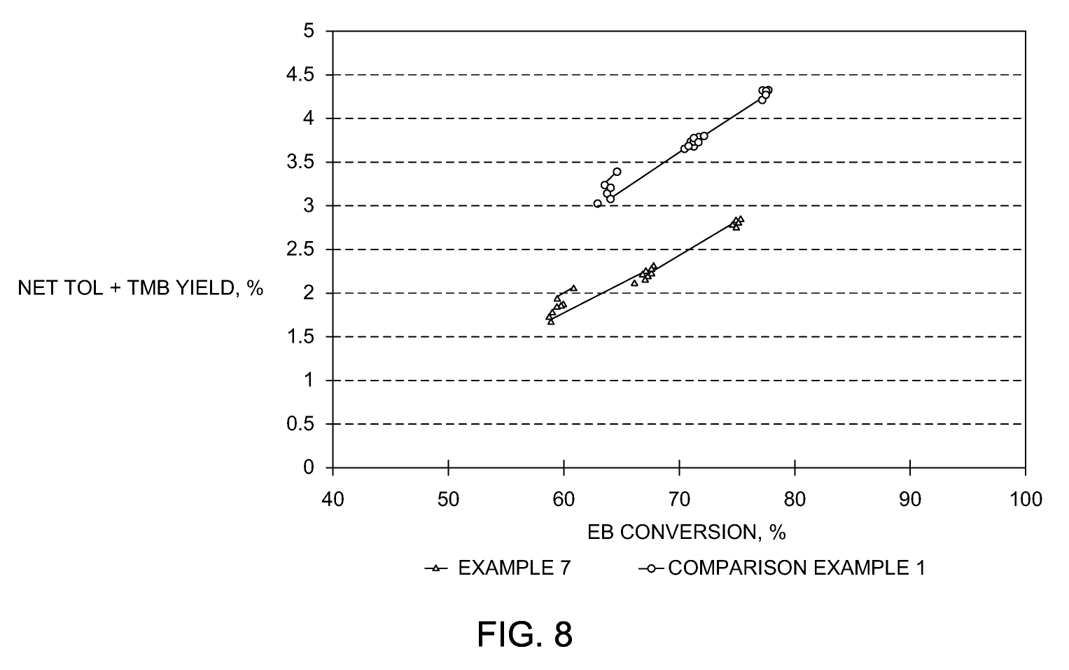

Referring to FIGS. 8-12, pilot plant tests are conducted with various supports or catalysts that include the supports impregnated with a catalytic metal. A gas chromatograph sold under the trade designation HP5890 by the Hewlett Packard Corporation of Palo Alto, Calif. is used to analyze the resulting products.

### Example 11

Referring to FIG. 8, a pilot plant test is conducted comparing the support of Example 7 to a support, i.e., a catalyst absent of any deposited metals, consisting of ion-exchanged and calcined steamed support prepared in the manner of Example 1 in U.S. Pat. No. 6,143,941 and indicated as Comparison Example 1. The molecular sieves and/or catalysts are tested under the same conditions with the same feeds for ethylbenzene conversion. As depicted, the support of Example 7 exhibits better selectivity and a good pX/X ratio. Moreover, no steaming is required and the pX/X ratio for the toluene disproportionation reaction is about 0.47, which is better than the support of Comparison Example 1.

### Example 12

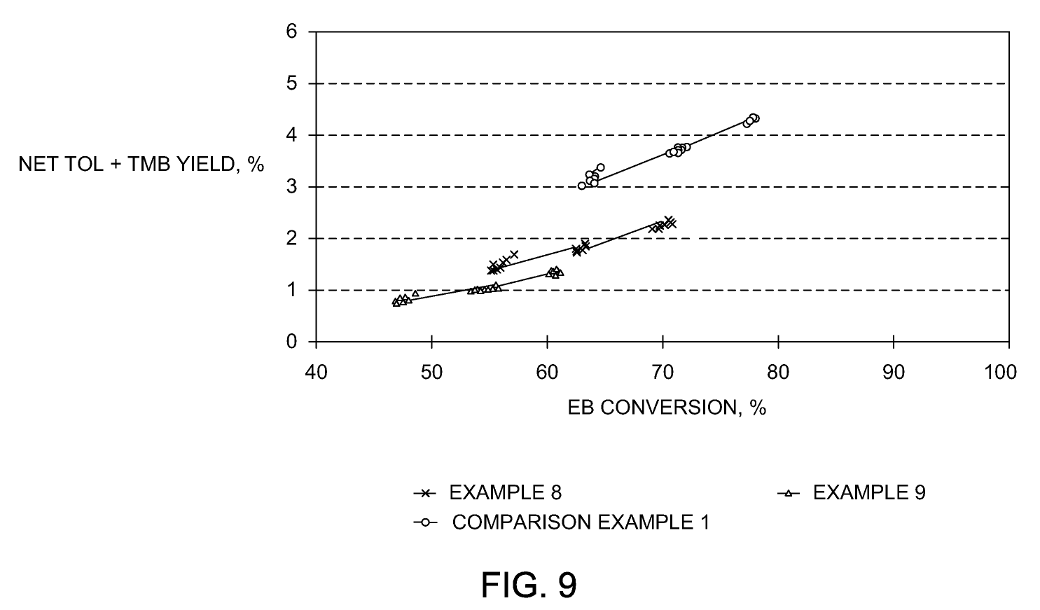

Referring to FIG. 9, an ethylbenzene dealkylation test is conducted comparing net TOL and TMB yield versus EB conversion in weight percent. The supports are tested under the same conditions with the same feeds. A support (may be referenced as Comparison Example 1) consisting of ion-exchanged and calcined steamed support prepared in the manner of Example 1 in U.S. Pat. No. 6,143,941, is compared to supports made by Examples 8 and 9.

As depicted, the Examples 8 and 9 demonstrate lower yields of TOL and TMB at a comparable EB conversion as compared to Comparison Example 1 without impregnation with a metal, such as platinum. As a consequence, the examples provide a significant and unexpected benefit of a support providing lower yields of TOL and TMB. Incorporating such a support into a catalyst can provide greater selectivity for a desired product and minimize unwanted side-reactions.

### Example 13

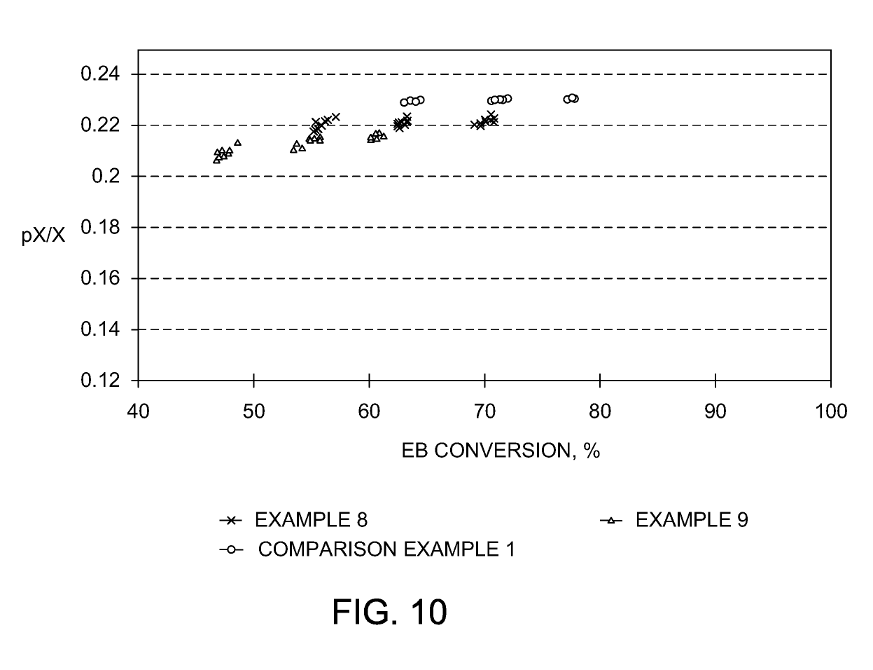

Referring to FIG. 10, another plant test is conducted with supports made according to Examples 8 and 9, and the support of Comparison Example 1, under the same conditions with the same feeds. Referring to FIG. 10, the pX/X ratio is lower for Examples 8 and 9 at a lower EB conversion.

### Example 14

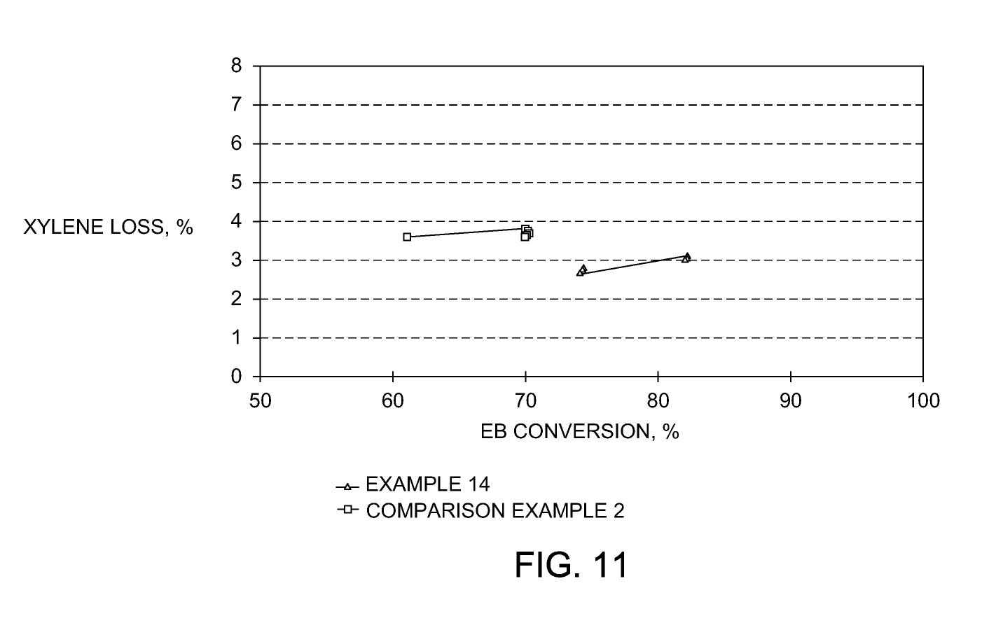

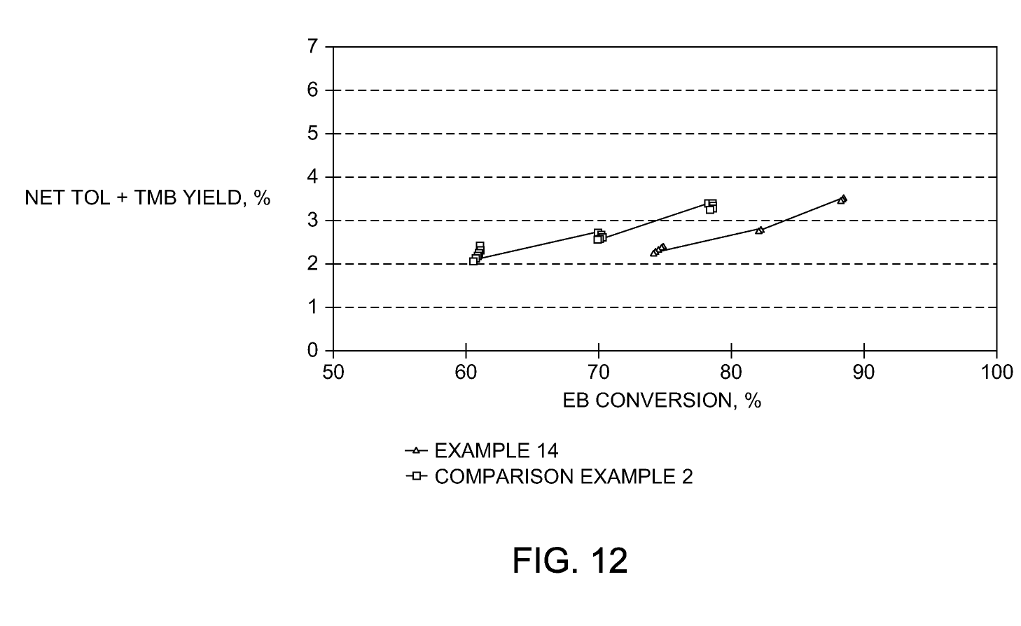

Referring to FIGS. 11 and 12, the support for Example 14 is made by combining 103.6 grams of zeolite made according to Example 1, 75 grams of silica sold under the trade designation LUDOX AS-40 by E. I. Du Pont De Nemours and Company of Wilmington, Del., 25.5 grams of precipitated silica sold under the trade designation HISIL by PPG Industries of Pittsburg, Pa., and 59.5 grams of distilled water in a mixer. Thus, the binder is a combination of commercially available silicas, namely a silica sold under the trade designation LUDOX AS-40 and a silica sold under the trade designation HISIL. For Comparison Example 2, the support includes 65%, by weight, MFI zeolite with a Si/Al₂ mole ratio of 80, 20%, by weight, HISIL silica, and 15%, by weight, LUDOX silica. For both Example 14 and Comparison Example 2, the resultant doughs are extruded into 0.16 centimeter trilobes. Afterwards, the extrudates are dried at 100° C. overnight and then calcined at 500° C. for six hours.

Both of these supports are impregnated with platinum by impregnating 111 grams of the calcined product with a 200 milliliters aqueous solution containing tetraammineplatinum chloride and 9.41 g ammonium nitrate followed by evaporation at 100° C., calcination at 582° C., ammonium nitrate ion exchange and oxidation at 543° C., so Comparison Example 2 has a platinum content of 0.05%, by weight, and Example 14 has a platinum content of 0.06%, by weight, based on the weight of the catalyst. Both catalysts of Example 14 and Comparison Example 2 are reduced in the lab at 425° C. for four hours prior to testing with a hydrogen to hydrocarbon ratio of 4, by weight, a pressure of about 800 kPa, and a weight hourly space velocity of 7. The test conditions are the same for both samples, namely converting a feed having 60% meta-xylene, 25% ortho-xylene, and 15% ethylbenzene, by weight.

Referring to FIG. 11, the xylene loss is lower at a higher EB conversion for Example 14 as compared to Comparison Example 2. Referring to FIG. 12, Example 14 provides higher EB conversion at similar net yield of TOL and TMB. Hence, the examples show a higher selectivity for EB conversion, which is significant and unexpected.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

From the foregoing description, one skilled in the art can easily ascertain the essential characteristics of this invention and, without departing from the spirit and scope thereof, can make various changes and modifications of the invention to adapt it to various usages and conditions.

1. A molecular sieve, comprising:
A) one or more crystals wherein the molecular sieve has an external surface area of no more than about 20 m²/g.

2. The molecular sieve according to claim 1, wherein the molecular sieve comprises a zeolite.

3. The molecular sieve according to claim 1, wherein the molecular sieve comprises an MFI zeolite.

4. The molecular sieve according to claim 3, wherein the external surface area is no more than about 12 m²/g.

5. The molecular sieve according to claim 3, wherein the external surface area is no more than about 8 m²/g.

6. A catalyst, comprising:
A) a molecular sieve comprising one or more crystals wherein the molecular sieve has an external surface area of no more than about 20 m²/g; and
B) a binder.

7. The catalyst according to claim 6, wherein the molecular sieve comprises an MFI zeolite.

8. The catalyst according to claim 7, wherein the binder comprises at least one of titania, zirconia, alumina, magnesia, thoria, beryllia, quartz, aluminum phosphate, and silica.

9. The catalyst according to claim 7, wherein the binder comprises alumina, aluminum phosphate, or silica.

10. The catalyst according to claim 7, wherein the molecular sieve comprises about 1-about 99%, by weight, based on the weight of the catalyst.

11. The catalyst according to claim 7, wherein the molecular sieve comprises about 25-about 75%, by weight, based on the weight of the catalyst.

12. The catalyst according to claim 7, wherein the external surface area is no more than about 12 m²/g.

13. The catalyst according to claim 7, wherein the external surface area is no more than about 8 m²/g.

14. A process for dealkylating ethylbenzene, comprising:
A) passing a stream comprising the ethylbenzene over an effective amount of a catalyst, wherein the catalyst comprises:
1) a molecular sieve comprising one or more crystals wherein the molecular sieve has an external surface area of no more than about 20 m²/g; and
2) a binder.

15. The process according to claim 14, wherein the molecular sieve comprises an MFI zeolite.

16. The process according to claim 15, wherein the binder comprises at least one of titania, zirconia, alumina, magnesia, thoria, beryllia, quartz, aluminum phosphate, and silica.

17. The process according to claim 15, wherein the binder comprises alumina, aluminum phosphate, or silica.

18. The process according to claim 15, wherein the molecular sieve comprises about 25-about 75%, by weight, based on the weight of the catalyst.

19. The process according to claim 15, wherein the external surface area is no more than about 12 m²/g.

20. The process according to claim 15, wherein the external surface area is no more than about 8 m²/g.
